(19) United States
(12) Patent Application Publication
(10) Pub. No.: US 2024/0406641 A1
(43) Pub. Date: Dec. 5, 2024

(54) HEARING AID EARPHONE AND GAIN PROCESSING METHOD AND DEVICE THEREFOR, AND READABLE STORAGE MEDIUM

(71) Applicant: GOERTEK INC., Weifang, Shandong (CN)

(72) Inventors: Ziwei Tong, Shandong (CN); Chunyan Liu, Shandong (CN)

(73) Assignee: GOERTEK INC., Weifang, Shandong (CN)

(21) Appl. No.: 18/695,220

(22) PCT Filed: Dec. 14, 2021
(86) PCT No.: PCT/CN2021/137867

§ 371 Date: Mar. 25, 2024

(30) Foreign Application Priority Data
Oct. 15, 2021 (CN) 202111204755.X

(51) Int. Cl. H04R 25/00

(52) U.S. Cl. CPC H04R 25/505; H04R 25/558; H04R 2225/41; H04R 2225/55; H04R 2430/01

(57) ABSTRACT

Disclosed are a gain processing method, device, and system for a hearing aid earphone, and a computer readable storage medium. The method comprises: acquiring raw user data of a user, the raw user data comprising initial hearing data; analyzing the raw user data by using a pre-established gain model to obtain gain data, the gain data comprising gain values respectively corresponding to different loudnesses under different frequencies; wherein, the gain model is established based on a plurality of user data historical samples collected in advance; and setting hearing assistive gain of the hearing assistive earphone based on the gain data.

### CROSS-REFERENCE TO RELATED APPLICATIONS

The present disclosure is a National Stage of International Application No. PCT/CN2021/137867, which claims priority to a Chinese patent application No. 202111204755.X filed with the CNIPA on Oct. 15, 2021 and entitled “hearing aid earphone and gain processing method and device therefor, and readable storage medium”, both of which are hereby incorporated by reference in their entireties.

### TECHNICAL FIELD

The present disclosure relates to the field of hearing aid, and particularly to a hearing aid earphone and a gain processing method, device and system therefor.

### BACKGROUND

Speech intensity typically ranges from 50 to 100 dB SPL. For a person with normal hearing, the speech intensity is within a dynamic range, wherein moderate speech intensity is the optimal threshold. However, for patients with sensorineural hearing loss with different hearing thresholds, they have different comfort zones for speech intensity, and need to use hearing aid earphone to hear the outside sound.

The gain of ordinary linear amplification hearing aid earphone is constant, that is, the gain provided is the same for all sound intensities, and is processed using clipping techniques only if the sound intensity is above the discomfort threshold. In general, WDRC parameters of the hearing aid earphone are tested by professional testers according to the preset calculation formula and cannot be configured individually according to the specific situation of a user, which leads to poor configuration accuracy and cannot provide a gain that is more in line with the needs of users, thereby negatively influencing user experience.

In view of the above, how to provide a hearing aid earphone and a gain processing method and a device therefor and a computer-readable storage medium capable of improving user's experience becomes a problem to be solved by those skilled in the art.

### SUMMARY

An objective of an embodiment of the present disclosure is to provide a hearing aid earphone, a gain processing method and a device therefor and a computer-readable storage medium, which can more accurately obtain the gain of meeting user's need during use and is conducive to improving the user's experience.

To solve the above technical problem, an embodiment of the present disclosure provides a gain processing method for a hearing aid earphone, comprising:
acquiring raw user data of a user, the raw user data comprising initial hearing data;
analyzing the raw user data by using a pre-established gain model to obtain gain data, the gain data comprising gain values respectively corresponding to different loudnesses under different frequencies; wherein, the gain model is established based on a plurality of user data historical samples collected in advance; and
setting hearing aid gain of the hearing aid earphone based on the gain data.

Optionally, a process of establishing the gain model is as follows:
analyzing a plurality of user data historical samples in a sample database by using a KCCA algorithm to obtain correlation coefficients corresponding to each of the user data historical samples;
selecting a maximum correlation coefficient from all correlation coefficients; and
establishing a gain model based on the maximum correlation coefficient.

Optionally, after said “acquiring raw user data of a user”, the method further comprising:
deleting abnormal hearing data in the raw user data to obtain processed user data; then, a process of said “analyzing the raw user data by using a pre-established gain model to obtain gain data” is as follows:
analyzing the processed user data by using the pre-established gain model to obtain the gain data.

Optionally, a process of said “deleting abnormal hearing data in the raw user data” is as follows:
converting the initial hearing data in the raw user data into hearing threshold data; and
deleting abnormal data in the hearing threshold data that is not within a preset range to obtain preprocessed user data.

Optionally, further comprising:
presenting the gain data through a terminal device so that the user adjusts the gain data;
receiving an adjustment instruction input by the user, and determining a target loudness and a target gain value based on the adjustment instruction; and
adjusting a gain value corresponding to the target loudness to the target gain value.

Optionally, further comprising:
taking adjusted gain data and user data corresponding to the user as a new user data historical sample;
adding the new user data historical sample to a sample database storing user data historical samples, and updating the sample database.

Optionally, the user data further comprises age, gender, and native place.

An embodiment of the present disclosure further provides a gain processing device for a hearing aid earphone, comprising:
an acquiring module configured for acquiring raw user data of a user, the raw user data comprising initial hearing data;
an analyzing module configured for analyzing the raw user data by using a pre-established gain model to obtain gain data, the gain data comprising gain values respectively corresponding to different loudnesses under different frequencies; wherein, the gain model is established based on a plurality of user data historical samples collected in advance; and
a setting module configured for setting hearing aid gain of the hearing aid earphone based on the gain data.

An embodiment of the present disclosure further provides a hearing aid earphone, comprising a left earphone, a right earphone, a memory and a processor, wherein:
the memory is configured for storing computer program; and
the processor is configured for implementing steps of the gain processing method for a hearing aid earphone as described above when executing the computer program.

An embodiment of the present disclosure further provides a computer readable storage medium, the computer readable storage medium stores a computer program therein, and the computer program, when executed by a processor, implements steps of the gain processing method for a hearing aid earphone as described above.

An embodiment of the present disclosure provides a gain processing method, device for a hearing aid earphone, a system and a computer readable storage medium, and the method comprises: acquiring raw user data of a user, the raw user data comprising initial hearing data: analyzing the raw user data by using a pre-established gain model to obtain gain data, the gain data comprising gain values respectively corresponding to different loudnesses under different frequencies: wherein, the gain model is established based on a plurality of user data historical samples collected in advance.

It can be seen that in the embodiment of the present disclosure, the gain data corresponding to the user may be obtained by analyzing the acquired raw user data through the pre-established gain model, and the gain data comprises gain values respectively corresponding to different loudnesses under different frequencies: by setting hearing aid gain of the hearing aid earphone based on the gain data, the earphone can output a sound that is more in line with the user's need when working under the hearing aid gain, and the present disclosure can more accurately obtain the gain of meeting user's need during use and is conducive to improving the user's experience.

### BRIEF DESCRIPTION OF THE DRAWINGS

In order to clearly illustrate technical solutions of the embodiments of the present disclosure or those in the prior art, accompanying drawings that need to be used in the embodiments or the prior art will be briefly introduced as follows. Obviously, drawings in following description are only a part of the present disclosure. For those skilled in the art, other drawings can also be obtained according to the disclosed drawings without creative efforts.

FIG. 1 shows a schematic flow diagram of a gain processing method for a hearing aid earphone provided by an embodiment of the present disclosure;

FIG. 2 shows a schematic illustration of a data transmission architecture provided by an embodiment of the present disclosure;

FIG. 3 shows a schematic illustration of an architecture of a gain model provided by an embodiment of the present disclosure:

FIG. 4 shows a schematic illustration of gain processing of a gain model provided by an embodiment of the present disclosure;

FIG. 5 shows a graph of output data corresponding to FIG. 4;

FIG. 6 shows a schematic illustration of gain processing for another gain model provided by an embodiment of the present disclosure:

FIG. 7 shows a graph of output data corresponding to FIG. 6:

FIG. 8 shows a schematic structural illustration of a gain processing device for a hearing aid earphone provided by embodiments of the present disclosure.

### DETAILED DESCRIPTION

Technical solutions in the embodiments of the present disclosure are described below with reference to the accompanying drawings in the embodiments of the present disclosure. Obviously, the described embodiments are merely a few of rather than all of the embodiments of the present disclosure. All other embodiments, acquired by those of ordinary skill in the art based on the embodiments of the present disclosure without any creative work, should fall into the protection scope of the present disclosure.

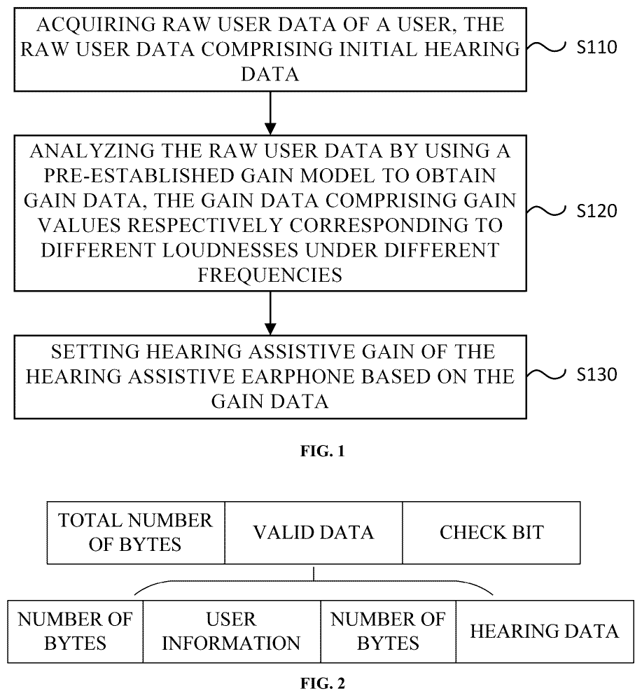

Please refer to FIG. 1, which shows a schematic flow diagram of a gain processing method for a hearing aid earphone provided by an embodiment of the present disclosure.
S110: acquiring raw user data of a user, the raw user data comprising initial hearing data:

It should be noted that, in the embodiment of the present disclosure, a large amount of user data is collected in advance, the user data is stored in a sample database as user data historical samples, and a gain model is established according to the user data historical samples, wherein the user data historical samples comprises hearing data and gain data corresponding to the user. In addition, in order to make the established gain model more optimized so that better gain data may be obtained through the gain model, the user data historical samples in the embodiment of the present disclosure may further comprise user information in addition to the hearing data and the gain data, and the user information may specifically include information such as age, gender, native place, and of course, user location information.

Specifically, for a user who needs to use the hearing aid earphone, before the user normally uses the hearing aid earphone, it is necessary to determine the hearing aid gain demanded by the user, and it is specifically possible to obtain raw user data of the user, wherein the raw user data comprises the user's initial hearing data, which may be obtained by conducting a hearing test on the user using the hearing aid earphone. Of course, the raw user data in the corresponding embodiment of the present disclosure may further comprise information such as age, gender, native place, and the like of the user in addition to the initial hearing data.
S120: analyzing the raw user data by using a pre-established gain model to obtain gain data, the gain data comprising gain values respectively corresponding to different loudnesses under different frequencies: wherein, the gain model is established based on a plurality of user data historical samples collected in advance:

It should be noted that after the raw user data of the user is obtained, the raw user data is input into the pre-established gain model, and the raw user data is analyzed through the gain model to obtain the corresponding gain data.
S130: setting hearing aid gain of the hearing aid earphone based on the gain data.

Specifically, the gain data in the embodiment of the present disclosure corresponds to the personal situation of the user, and it is possible to obtain a better hearing aid effect after the hearing aid gain of the hearing aid earphone is adjusted through the gain data, such that the sound output by the hearing aid earphone better meets the user's own needs.

Further, after obtaining the raw user data of the user in above S110, the method may further comprise:
deleting abnormal hearing data in the raw user data to obtain processed user data:

It should be noted that in a practical application, the raw user data transmitted by the client can be accepted first. Specifically, the data can be received according to the data format shown in FIG. 2, and of course, the data can be received by sampling XML or custom format, and the raw user data can be transmitted in an encrypted form during the transmission so as to prevent data leakage. For example, in this case, transmission is carried out in a symmetric encrypted or asymmetric encrypted manner, and, of course, transmission in a non-encrypted form can also be carried out, which can be determined specifically according to the actual need, and the embodiment of the present disclosure do not make any special limitations thereon.

Specifically, after receiving the user's raw user data, the raw user data may be preprocessed in order to delete the abnormal data and obtain the processed user data, so that the subsequent sampling pre-established gain model analyzes the processed user data to obtain the gain data corresponding to the user, so that the gain data obtained is more accurate and better meets the user's personal needs.

Wherein, a process of preprocessing is as follows:
converting the initial hearing data in the raw user data into hearing threshold data (loudness that can be heard at different frequencies): deleting abnormal data in the hearing threshold data that is not within a preset range to obtain preprocessed user data. Wherein, if the hearing threshold data is less than −10, the value is −10, and if the hearing threshold data is greater than 100, the value is 100, thereby obtaining valid two-dimensional data. It should be noted that the user data historical samples is also the processed user data, and this preprocessing is to process the raw user data to have the same dimensions and parameters as the user data historical samples.

Further, the method may further comprise:
presenting the gain data through a terminal device so that the user adjusts the gain data;
receiving an adjustment instruction input by the user, and determining a target loudness and a target gain value based on the adjustment instruction; and
adjusting a gain value corresponding to the target loudness to the target gain value.

It should be noted that in order to further meet the personalized needs of the user to better meet the user's needs, in the embodiment of the present disclosure, after the gain data is obtained, it may also be presented to the user through the terminal device. Specifically, gain values at different frequencies (e.g., 125, 250, 500, 1000, 2000, 4000, 8000, etc.) and of different loudness (e.g., 20 db, 35 db, 50 db, 65 db, 80 db, 95 db, etc.) are all presented to the user, and the user can adjust the displayed gain data according to his own needs, such as adjusting the gain value corresponding to a certain loudness at a certain frequency, such that after the gain processing is carried out on the corresponding loudness at the frequency according to the adjusted gain value, the user can hear the corresponding sound better and more comfortably. Specifically, the gain data may be presented through the APP interface of the mobile terminal (mobile phone) or the display interface of the computer, and the user may adjust the gain data through the APP interface of the mobile terminal (mobile phone) or the display interface of the computer, so as to obtain the most suitable gain value for the user.

Further, the method may further comprise:
taking adjusted gain data and user data corresponding to the user as a new user data historical sample;
adding the new user data historical sample to a sample database, and updating the sample database.

It should be noted that in the embodiment of the present disclosure, after the user adjusts the gain data, the adjusted gain data combined with the corresponding user data is added to the sample database as a new user data historical sample as a whole, and therefore the sample database is improved continuously such that a better deep learning gain module is obtained when the model is trained through the user data historical samples in the sample database.

Further, a process of establishing the above gain model may be as follows:
analyzing a plurality of user data historical samples in a sample database by using a KCCA algorithm to obtain correlation coefficients corresponding to each of the user data historical samples;
selecting a maximum correlation coefficient from all correlation coefficients;
establishing a gain model based on the maximum correlation coefficient.

It should be noted that each user data historical sample comprises user sample data, hearing sample data, and gain sample data. By taking the user sample data and the hearing sample data as input data, taking the corresponding gain data as output data, and analyzing each group of input data and output data by using the KCCA algorithm, the correlation coefficient of each group of input data and the corresponding output data is obtained so as to obtain a plurality of groups of correlation coefficients, and then finding out the maximum correlation coefficient from the plurality of groups of correlation coefficients so as to establish the gain model by using the maximum correlation coefficient.

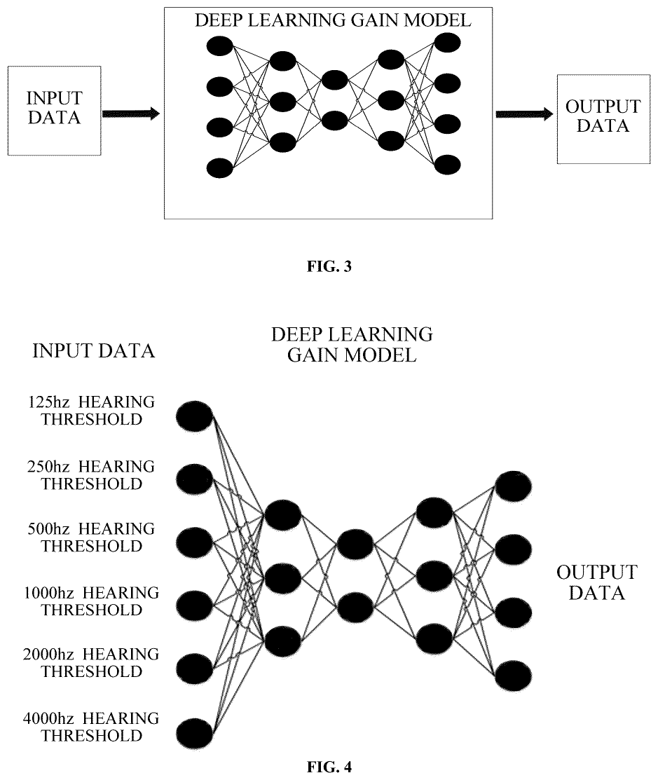

Specifically, the deep learning gain module is established according to the maximum correlation coefficient, and as shown in FIG. 3, the raw user data of the user is input into the gain model to obtain output data, which is the gain data corresponding to the user.

Specifically, the user sample data and the hearing sample data may be used as the input data, wherein the user sample data mainly refers to the user's environmental noise, age, gender, native place and other data, which is equivalent to the user's basic information: the hearing sample data refers to the user's gain data, that is, different users may have different gain data in different environments. The input data is taken as sample X, the corresponding gain data is taken as the output data, and the output data is taken as sample Y, and the kernel matrices K_{X} and K_{Y} are defined and calculated by using the samples X, Y and a kernel function K(x,z), wherein the kernel function is:

$${K}_{( x , z )} = {e}^{- \frac{{\vert x - z \vert}^{2}}{2 {σ}^{2}}} ,$$

x,z∈X, X belongs to R(n) space, n is the space dimension, x and z represent any two elements in the input sample, and σ represent the hyper-parameter of the RBF kernel.

M, L and N are calculated according to expressions

$$M = \frac{1}{N} {K}_{X}^{T} {JK}_{Y} , \qquad \begin{matrix}L = \frac{1}{n} {K}_{X}^{T} {JK}_{X} + η {K}_{X} , & N = \frac{1}{n} {K}_{Y}^{T} {JK}_{Y} + η {K}_{Y} ,\end{matrix}$$

wherein J=I−λλ^{T}, λ=(1, . . . , 1)^{T}, I represents a unit vector, η represents a constant, and by this constraint, a meaningful canonical variable is generated in the high-dimensional feature space, wherein K_{X}=ΦX′ΦX, ΦX, are vector forms of input data.
α and β are further calculated according to expressions L⁻¹MN⁻¹M^{T}α=λ²α and N⁻¹M^{T}L⁻¹Mβ=λ²β, wherein λ represents a unit vector.
   μ and ν are further calculated according to expressions

$$μ = {\sum }_{i = 1}^{{N}^{′}} {α}_{i} {K}_{x} ( {X}_{i} , X ) and v = {\sum }_{i = 1}^{{N}^{′}} {β}_{i} {K}_{Y} ( {Y}_{i} , Y ) ,$$

and represents projections of high-dimensional feature spaces on vectors, respectively, wherein αᵢ is the ith element of the vector α, βᵢ is the ith element of the vector β, N′ represents a total number of elements of vectors α and β, and both of Kₓ(Xᵢ,X) and K_{y}(Yᵢ,Y) represent the nuclear matrices, Xᵢ represents the ith element of the vector X, and Yᵢ represents the ith element of the vector Y. The correlation coefficient ρ(μ,ν) is further calculated according to μ and ν, wherein:

$$ρ ( μ , v ) = \frac{{\sum }_{i = 1}^{N} {u}_{i} {v}_{i} - \frac{{\sum }_{i = 1}^{N} {u}_{i} {\sum }_{i = 1}^{N} {v}_{i}}{N}}{\sqrt{( {\sum }_{i = 1}^{N} {u}_{i} {u}_{i} - \frac{{( {\sum }_{i = 1}^{N} {u}_{i} )}^{2}}{N}} ) ( {\sum }_{i = 1}^{N} {v}_{i} {v}_{i} - \frac{{( {\sum }_{i = 1}^{N} {v}_{i} )}^{2}}{N} )} ,$$

wherein μᵢ represents the ith element of the vector μ, and νᵢ represents the ith element of the vector ν.

For each group of input data and output data, the corresponding correlation coefficient ρ(μ,ν) is calculated by the above calculation process, and then the maximum correlation coefficient is selected from these correlation coefficients.

After calculating the correlation coefficient, the gain model is constructed as follows.

By inputting a user sample data X, the gain data vector Y may be obtained by a formula related to the maximum correlation coefficient ρ as follows (including but not limited to this linear way).

Y=ρ1*X1+ρ2*X2+ . . . +ρn*Xn; wherein ρ1 is the correlation coefficient between X1 and Y (gain data), and ρn is the correlation coefficient between Xn and Y (gain data).

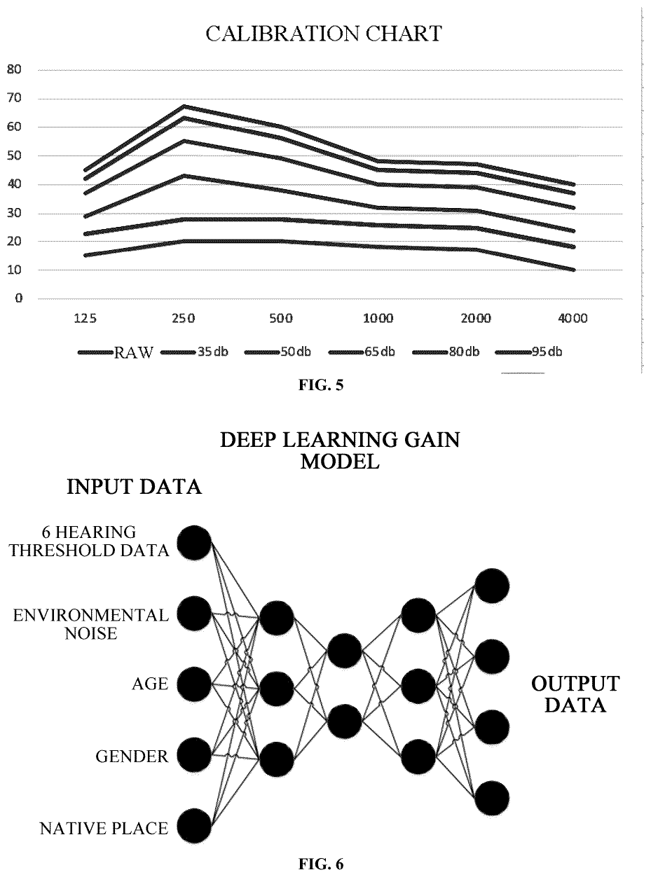

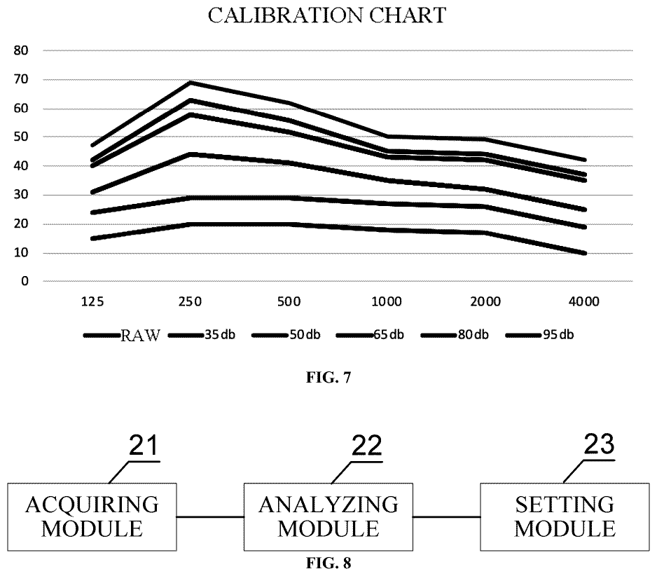

In addition, as shown in FIGS. 4-7, for the gain data obtained by the user at 40 db of noise, wherein the hearing threshold data of the user is taken as the input data in FIG. 4, and the obtained output data is shown in FIG. 5 and table 1: the user data (such as age, gender, native place, etc.) and the hearing threshold data of the user are taken as output data, and the obtained output data is shown in FIG. 7 and table 2. It can be seen that with more training inputs, the resulting gain data has better gain for the patient. Wherein:

TABLE 1

| | 125 | 250 | 500 | 1000 | 2000 | 4000 |
|---|---|---|---|---|---|---|
| Raw | 42 | 63 | 56 | 45 | 44 | 37 |
| 35 db | 37 | 55 | 49 | 40 | 39 | 32 |
| 50 db | 45 | 67 | 60 | 48 | 47 | 40 |
| 65 db | 29 | 43 | 38 | 32 | 31 | 24 |
| 80 db | 23 | 28 | 28 | 26 | 25 | 18 |
| 95 db | 15 | 20 | 20 | 18 | 17 | 10 |

TABLE 2

| | 125 | 250 | 500 | 1000 | 2000 | 4000 |
|---|---|---|---|---|---|---|
| Raw | 42 | 63 | 56 | 45 | 44 | 37 |
| 35 db | 40 | 58 | 52 | 43 | 42 | 35 |
| 50 db | 47 | 69 | 62 | 50 | 49 | 42 |
| 65 db | 31 | 44 | 41 | 35 | 32 | 25 |
| 80 db | 24 | 29 | 29 | 27 | 26 | 19 |
| 95 db | 15 | 20 | 20 | 18 | 17 | 10 |

It can be seen that in the embodiment of the present disclosure, the gain data corresponding to the user may be obtained by analyzing the acquired raw user data through the pre-established gain model, and the gain data comprises gain values respectively corresponding to different loudnesses under different frequencies: by setting hearing aid gain of the hearing aid earphone based on the gain data, the earphone can output a sound that is more in line with the user's need when working under the hearing aid gain, and the present disclosure can more accurately obtain the gain of meeting user's need during use and is conducive to improving the user's experience.

On the basis of the above embodiment, an embodiment of the present disclosure further provides a gain processing device for a hearing aid earphone, and specifically referring to FIG. 8, the device comprises:
an acquiring module 21 configured for acquiring raw user data of a user, the raw user data comprising hearing data;
an analyzing module 22 configured for analyzing the raw user data by using a pre-established gain model to obtain gain data, the gain data comprising gain values respectively corresponding to different loudnesses under different frequencies: wherein, the gain model is established based on a plurality of user data historical samples collected in advance;
a setting module 23 configured for setting hearing aid gain of the hearing aid earphone based on the gain data.

It should be noted that the gain processing device for a hearing aid earphone in the embodiment of the present disclosure has the same beneficial effects as the gain processing method for a hearing as provided in the above embodiment, and please refer to the above embodiment for a detailed introduction of the gain processing method for a hearing aid earphone involved in the embodiment of the present disclosure, which will not be repeated herein.

On the basis of the above embodiment, an embodiment of the present disclosure further provides a hearing aid earphone, comprising a left earphone, a right earphone, a memory and a processor, wherein:
the memory is configured for storing computer program;
the processor is configured for implementing steps of the gain processing method for a hearing aid earphone as described above when executing the computer program.

For example, the processor in the embodiment of the present disclosure is specifically configured for: acquiring raw user data of a user, the raw user data comprising initial hearing data: analyzing the raw user data by using a pre-established gain model to obtain gain data, the gain data comprising gain values respectively corresponding to different loudnesses under different frequencies: wherein, the gain model is established based on a plurality of user data historical samples collected in advance: setting hearing aid gain of the hearing aid earphone based on the gain data.

On the basis of the above embodiment, an embodiment of the present disclosure further provides a computer readable storage medium, the computer readable storage medium stores computer program therein, and the computer program, when executed by a processor, implements steps of the gain processing method for a hearing aid earphone as described above.

The computer readable storage medium may comprise: U disk, mobile hard disk, read-only memory (ROM), random access memory (RAM), disk or CD and other medium that may store program code.

Each embodiment in this specification is described in a progressive manner, and each embodiment focuses on its differences from other embodiments, and the similar parts between each embodiment can refer to each other. For the device disclosed in the embodiments, the description is relatively simple because the device corresponds to the method disclosed in the embodiments, and the relevant points can be found in the description of the method.

It would also be understood by one of ordinary skill in the art that the units and algorithmic steps of the various examples described in conjunction with the embodiments disclosed herein are capable of being implemented in electronic hardware, computer software, or a combination of both, and to clearly illustrate the interchangeability of hardware and software, the composition and steps of the various examples have been described in the foregoing description in general terms according to function. Whether these functions are performed in hardware or software depends on the particular application and design constraints of the technical solution. Those skilled in the art may use different methods to implement the described functions for each particular application, but such implementation should not be considered beyond the scope of the present disclosure.

The steps of the methods or algorithms described in conjunction with the embodiments disclosed herein may be implemented directly with hardware, a software module executed by a processor, or a combination of both. The software module may be placed in random memory (RAM), memory, read-only memory (ROM), electrically programmable ROM, electrically erasable programmable ROM, registers, hard disks, removable disks, CD-ROMs, or any other form of storage medium known in the art

It should also be noted that in this article, relational terms such as “first” and “second”, etc., are used solely to distinguish one entity or operation from another, and do not necessarily require or imply any such actual relationship or sequence between those entities or operations. Further, the term “comprise”, “include” or any other variation thereof is intended to cover non-exclusive comprising so that a process, method, article or apparatus that comprises a series of elements includes not only those elements, but also other elements that are not expressly listed, or also comprises elements inherent in such process, method, article or apparatus. Without further limitation, the elements defined by the phrase “comprising a . . . ” do not preclude the existence of other identical elements in the process, method, article or apparatus that includes the elements.

1. A gain processing method for a hearing aid earphone, comprising:
acquiring raw user data of a user, the raw user data comprising initial hearing data;
analyzing the raw user data by using a pre-established gain model to obtain gain data, the gain data comprising gain values respectively corresponding to different loudness levels under different frequencies: wherein, the gain model is established based on a plurality of user data historical samples collected in advance; and
setting a hearing aid gain of the hearing aid earphone based on the gain data.

2. The gain processing method for a hearing aid earphone according to claim 1, wherein the gain model is established as follows:
analyzing a plurality of user data historical samples in a sample database by using a KCCA algorithm to obtain correlation coefficients corresponding to each of the user data historical samples;
selecting a maximum correlation coefficient from all correlation coefficients; and
establishing the gain model based on the maximum correlation coefficient.

3. The gain processing method for a hearing aid earphone according to claim 1, wherein after said acquiring raw user data of a user, the method further comprising:
deleting abnormal hearing data in the raw user data to obtain processed user data; then, said analyzing the raw user data by using a pre-established gain model to obtain gain data comprises:
analyzing the processed user data by using the pre-established gain model to obtain the gain data.

4. The gain processing method for a hearing aid earphone according to claim 3, wherein said deleting abnormal hearing data in the raw user data comprises:
converting the initial hearing data in the raw user data into hearing threshold data; and
deleting abnormal data in the hearing threshold data that is not within a preset range to obtain preprocessed user data.

5. The gain processing method for a hearing aid earphone according to claim 1, further comprising:
presenting the gain data through a terminal device so that the user adjusts the gain data;
receiving an adjustment instruction input by the user, and determining a target loudness and a target gain value based on the adjustment instruction; and
adjusting a gain value corresponding to the target loudness to the target gain value.

6. The gain processing method for a hearing aid earphone according to claim 5, further comprising:
taking adjusted gain data and user data corresponding to the user as a new user data historical sample; and
adding the new user data historical sample to a sample database storing user data historical samples, and updating the sample database.

7. The gain processing method for a hearing aid earphone according to claim 1, wherein the user data comprises age, gender, and native place.

8. A gain processing device for a hearing aid earphone, comprising:
an acquiring module configured for acquiring raw user data of a user, the raw user data comprising initial hearing data;
an analyzing module configured for analyzing the raw user data by using a pre-established gain model to obtain gain data, the gain data comprising gain values respectively corresponding to different loudness levels under different frequencies; wherein, the gain model is established based on a plurality of user data historical samples collected in advance; and
a setting module configured for setting hearing aid gain of the hearing aid earphone based on the gain data.

9. A hearing aid earphone, characterized by comprising a left earphone, a right earphone, a memory and a processor, wherein:
the memory is configured for storing a computer program; and
the processor is configured for implementing a gain processing method for a hearing aid earphone according to claim 1 when executing the computer program.

10. A computer readable storage medium, wherein the computer readable storage medium stores a computer program therein, and the computer program is executed by a processor to implement a gain processing method for a hearing aid earphone according to claim 1.
